(19) United States
(12) Patent Application Publication
(10) Pub. No.: US 2018/0064463 A1
(43) Pub. Date: Mar. 8, 2018

(54) DUAL END SYSTEMS AND METHODS FOR CROSSING AND TREATING AN OCCLUSION

(71) Applicant: XableCath Inc., Salt Lake City, UT (US)

(72) Inventors: Lisa K. Jordan, Salt Lake City, UT (US); Enora S. Rogers, Salt Lake City, UT (US)

(21) Appl. No.: 15/551,824

(22) PCT Filed: Feb. 16, 2016
(86) PCT No.: PCT/US2016/018126

§ 371 Date: Aug. 17, 2017

Related U.S. Application Data
(60) Provisional application No. 62/117,892, filed on Feb. 18, 2015.

(51) Int. Cl. A61B 17/3207; A61M 25/00

(52) U.S. Cl. CPC A61B 17/320758; A61B 2017/00853; A61M 25/0045

(57) ABSTRACT

A catheter device is provided that has a first end and a second end. The first end has a first rigid ring disposed thereon. The first rigid ring has an occlusion engaging feature disposed at a free end thereof. The second end has a second rigid ring disposed thereon. The second rigid ring has an occlusion engaging feature disposed at a free end thereof An elongate body extends between the first end and the second end. The elongate body has a lumen extending therethrough. A handle is configured such that a body thereof can be removeably coupled with the elongate body at a first position adjacent to the first end. The handle is configured such that the body thereof can be removeably coupled with the elongate body at a second position adjacent to the second end.

### BACKGROUND OF THE INVENTION

### Field of the Invention

This application is directed to systems and methods for treating occlusions, including crossing narrow passages of lumen segments or total occlusions.

### Description of the Related Art

A variety of techniques exist to de-bulk occluded vessel segments. While these techniques have met varying degrees of success, not all patients are successfully treated in this manner. Some patients with peripheral occlusions are left with few options other than amputation of the limb fed by the occluded artery. Such drastic techniques are obviously not available to patients with extensive occlusion of coronary and other critical arteries.

There are a number of products on the market that are designed for crossing CTOs. Intralumenal devices are one class of such products that in theory may reduce the dissection plane of a long occlusive lesion, protect collaterals and keep treatment options open.

### SUMMARY OF THE INVENTION

However, intraluminal products have operational challenges. For instance, if the matter making up the occlusion is to be ingested into the clearing device, a limitation is the volume of material that can be retained in the device. Also, the morphology of the material to be removed may indicate different cutting and/or supporting strategies. Accordingly, it would be useful to have a device that can be revised or the configuration of which can be selected by the clinician just before or during the procedure.

In one embodiment, a catheter device is provided that has a first end and a second end. The first end has a first rigid ring disposed thereon. The first rigid ring has an occlusion engaging feature disposed at a free end thereof. The second end has a second rigid ring disposed thereon. The second rigid ring has an occlusion engaging feature disposed at a free end thereof. An elongate body extends between the first end and the second end. The elongate body has a lumen extending therethrough. A handle is configured such that a body thereof can be coupled with the elongate body at a first position adjacent to the first end. In this configuration, the second end can be advanced into a patient. The inner diameter of the second rigid ring configured for sliding and supporting interaction with a guidewire. The handle is configured such that a body thereof can be coupled with the elongate body at a second position adjacent to the second end. In this configuration, the first end can be advanced into a patient. The inner diameter of the first rigid ring is configured for sliding and supporting interaction with a guidewire.

In another embodiment, a method is provided for enhancing access across an occlusion. A first end of a catheter is advanced into a blood vessel and up to a proximal face of an occlusion. The size of a lumen across at least a portion of an occlusion is expanded by engaging the first end of the catheter with the occlusion. The catheter is removed from the patient. A second end of the catheter opposite the first end is advanced into the blood vessel and up to the proximal face of the occlusion. The size of a lumen across at least a portion of the occlusion is further expanded by engaging the second end of the catheter with the occlusion.

In another embodiment, a dual end occlusion crossing device is provided. The device includes a first end that has a first occlusion engaging feature disposed at a free end thereof. The device includes a second end having a second occlusion engaging feature disposed at a free end thereof. An elongate body extends between the first end and the second end. The elongate body has a lumen that extends therethrough.

The occlusion engaging features can include sharp and/or abrasive features.

The dual end occlusion crossing device can be converted to permit either end to be inserted into the patient to act on an occlusion. A stop device can be moved, e.g., slid, along the elongate body to provide a positive stop preventing the dual end occlusion crossing device from sliding completely into the patient.

### BRIEF DESCRIPTION OF THE DRAWINGS

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIG. 1 illustrates schematically a near total occlusion;

FIG. 2 illustrates a system the can be used to provide access across an occlusion for therapy devices to enhance treatment of an occlusion:

FIG. 3 is a perspective view of a first embodiment of a device that can be used in the system of FIG. 2 in providing access across an occlusion for therapy devices;

FIG. 4 is a plan view of a second embodiment of a device for providing access across an occlusion for therapy devices;

FIG. 5 is an exploded perspective view of the second embodiment of a device of FIG. 4;

FIG. 6 is a perspective detail view of a distal portion of one variation of an occlusion crossing device, which can be incorporated into various embodiments including the first or second embodiments;

FIG. 7 is a perspective detail view of a distal portion of another variation of an occlusion crossing device, which can be incorporated into various embodiments including the first or second embodiments;

FIG. 8 is a dual end occlusion device that can be reversed without removing a handle;

FIG. 8A shows a sliding retention mechanism;

FIG. 8B shows a blood loss minimizing feature that can be disposed in a lumen of the device of FIG. 8.

### DETAILED DESCRIPTION OF THE PREFERRED EMBODIMENT

Each and every feature described herein, and each and every combination of two or more of such features, is included within the scope of the present invention provided that the features included in such a combination are not mutually inconsistent.

Embodiments of the present invention are generally directed to catheter systems for crossing vascular stenosis, such as near total occlusions, components thereof, and methods use of such systems and components.

As used herein, the term “near total occlusion” refers to regions of vascular stenosis that reduce the cross-sectional area of the vessel lumen by >80%, in particular, by >90%, and in some cases by more than 95%. The term “total occlusion” means the entire vessel lumen is fully occupied by atheroma or other occlusive material preventing blood flow through the passage of the lumen.

As used herein, the term “substantially”, when used in reference to a linear dimension (e.g., length, width, thickness, distance, etc.) means within plus or minus one percent (1%) of the value of the referenced linear dimension.

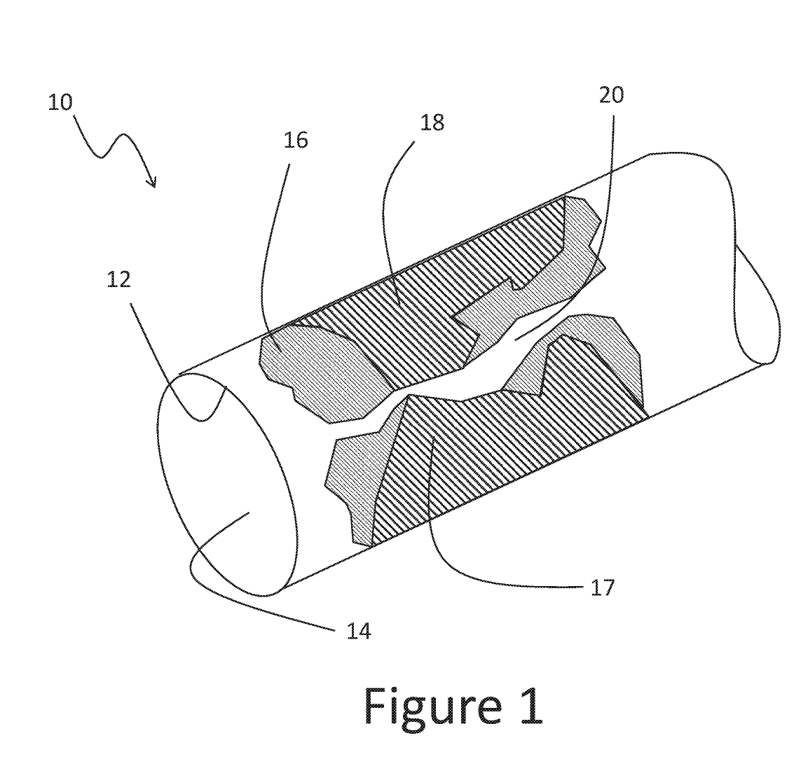

FIG. 1 illustrates a near total occlusion of a blood vessel formed by a lesion 17. The blood vessel 10 has an interior surface 12, which defines a lumen 14. In atherosclerosis, lipid and fibro muscular material accumulate in the vessel wall, forming a lesion that bulges into and occupies or occludes at least a portion of the lumen 14. Advanced-stage atherosclerotic lesions often include regions of soft plaque 16 and regions of atheroma 18. In some cases, the atheroma 18 may be calcified making access by interventional techniques difficult or impossible.

When the atheroma 18 intrudes into the lumen 14, a stenosis 20 is formed that can greatly reduce blood flow through the vessel. Angioplasty is one technique for treating a stenosis 20. In balloon angioplasty, a deflated balloon is mounted on an endovascular catheter, and the catheter is pushed along the vessel 10 until the deflated balloon occupies at least a portion of the stenosis 20. Once the deflated balloon is positioned within the stenosis 20, the balloon is inflated, pushing the atheroma 18 back toward the vessel wall and enlarging the lumen 14 within the region of stenosis 20. In some cases, an expandable stent is used to restore the lumen 14 within the region of stenosis 20.

In many cases, a guidewire is pushed ahead of the endovascular catheter to aid catheter travel through the blood vessel. The guidewire is thin and has a smaller profile than the catheter. Often, the catheter has a central lumen that accommodates the guidewire, and the catheter rides along the guidewire. This configuration of catheter is referred to as an “over-the-wire” catheter.

In some cases, the stenosis 20 is so narrow that the balloon catheter is unable to follow the guidewire through the stenosis. Rather, the balloon catheter can get hung-up or blocked at the proximal or distal end (depending on the direction of approach) of the stenosis 20. In such a case, angioplasty is precluded because it is not possible to position a deflated balloon within the stenosis 20. In some cases, the atheroma 18 forms a calcified plug that precludes passage of the guidewire through the stenosis 20.

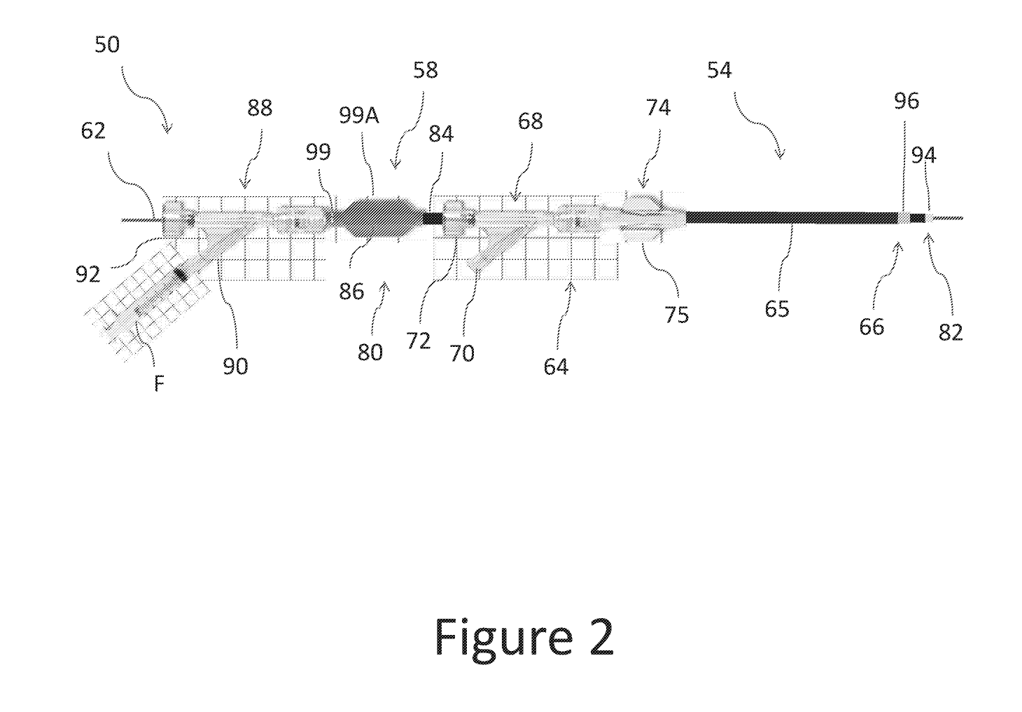

FIG. 2 illustrates an occlusion crossing system 50 that can be used to improve a clinician's ability to pass a balloon catheter or other therapy device across a blockage formed by the lesion 17. The occlusion crossing system 50 includes a sheath 54 and a catheter device 58. The catheter device 58 is provided for clearing a passage through the lesion 17 to enlarge the access therethrough, which may involve cutting the occlusion. For this reason, in some passages the catheter device 58 is referred to as a cutting catheter. The sheath 54 can be used to enclose and/or guide the catheter device 58 between a vascular access site and an occlusion. The sheath 54 thus provides protection for the un-occluded vessel(s) through which the catheter device 58 is delivered. The occlusion crossing system 50 can also include a guidewire 62 to help access or cross an occlusion.

The guidewire 62 can take any suitable form. It can be a long slender wire with no lateral protuberances or it can have one or more lateral extensions. For example a plurality of barriers or shoulders can be provided along a distal length of the guidewire 62 to engage and retain portions of the lesion 17. The guidewire 62 can have an anchor, such as a helical structure adapted to be advanced rotationally into the lesion to engage and hold it. These are examples of structures that can positively engage and hold the lesion 17. When so engaged, these structures can provide a counter traction for holding the position of the lesion while catheter device 58 (or variant herein) is advanced into the lesion to enhance access across the lesion. Examples of barriers and anchor are discussed in U.S. Pat. No. 5,443,443 and U.S. Pat. No. 5,047,040, which are hereby incorporated by reference herein in their entirety.

The sheath 54 comprises a proximal end 64, a distal end 66, and a lumen extending through an elongate body 65 disposed between the ends 64, 66. The lumen is sized to receive the catheter device 58 as discussed further below. The proximal end 64 of the sheath 54 is preferably configured to be coupled with other devices. For example, a branched access port 68 can be provided at the proximal end 64. A first branch 70 can be provided to couple with a fluid source. A second branch 72 can be aligned with the lumen of the sheath 54 to provide in-line access to the lumen of the sheath 54. One or both of the branches 70, 72 can have a valve structure to limit, minimize or eliminate blood loss. A tuohy-borst attachment can be provided on one or both of the branches 70, 72. In one embodiment, the proximal end 64 includes a modular coupling 74 that enables the branched access port 68 to be decoupled from the elongate body 65 if access via the branches is not required or for certain phases of procedures where the branches are not needed and might be in the way if not removed from the procedure zone. The coupling 74 can include torque structures 75 on opposite sides thereof.

The catheter device 58 is configured to be advanced to the occlusion 20 to provide a therapy as discussed herein. The catheter device 58 comprises a proximal end 80, a distal end 82, and a lumen extending through an elongate body 84 disposed between the ends 80, 82. The lumen is sized to provide access for a balloon catheter or other therapy device, for fluid to be injected or withdrawn, and/or for material of the occlusion 20 to be lodged. The elongate body 84 has sufficient rigidity for deliverability and for providing cutting or segmenting action at the occlusion 20. For example the body 84 can be configured to provide 1:1 torque. As discussed below, braids and coils are contemplated as structures providing pushability and flexibility for various applications, including peripheral, coronary and neuro-vascular applications.

The elongate body 84 has a length sufficient to reach a treatment site such as a peripheral, coronary, or neuro-vascular treatment site. For example, for ipsa-lateral treatment, the elongate body 84 can be between about 40 and about 100 cm, e.g., about 80 cm. For a treatment in the iliac artery, the elongate body 84 can be about 60 cm. For a treatment in the superficial femoral artery (SFA), the elongate body 84 can be between about 140 and 160 cm. For a treatment in the coronary arteries, the elongate body 84 can be between about 110 cm and about 140 cm. For neurovascular applications the elongate body 84 can be between about 130 cm and about 180 cm, e.g., about 150 cm. The sheath 54 can be about 10 cm to about 20 cm shorter than the catheter device 58. The elongate body 65 can be 10-20 cm shorter than the elongate body 84. More generally, the sheath 54 or elongate body 65 can be shorter than the catheter device 58 or elongate body 84 by an amount sufficient to provide a working length.

The length of the elongate body 84 can also be a function of the path to be traversed by the catheter device 58 to treat the patient. The access point for inserting the catheter device 58 can be in the groin on the patient, in the arm of the patient, or in the lower leg of the patient. Access at the groin can be at a femoral artery or vein. Access in the arm can be at a radial artery or vein. Access in the lower leg can be at a pedal artery or vein. Treatment site can be anywhere in the body that occlusions may form and where such occlusions provide risk to viable tissues. A catheter length of 200 cm or more can be used to traverse from the pedal artery to neurovaculature. A catheter length of 200 cm or more can be used to traverse from the radial artery to vessels below the ankle. A shorter length of approximately 150 cm can be used to reach the neurovaculature from the groin. A shorter length of approximately 110-120 cm can be used to reach the coronary vasculature from the groin. Still shorter lengths, e.g., 80-90 cm can be used to access vasculature of the foot from the pedal artery or from the groin.

For longer catheters, the elongate body 84 can be configured to facilitate access to remote vessels. For instance, the stiffness of the body can be tailored to sustain substantially 1:1 toque. A proximal zone can include a stiff structure, such as a hypotube. A zone distal the proximal zone can be more flexible. For instance a continuous change in a support member such as a braid can make the elongate body 84 progressively more flexible toward the distal end. Also, the wall thickness and or diameter of the elongate body 84 can be reduced toward the distal end. As discussed above, the tip structure, e.g., the occlusion clearing implement 94 can be much stiffer than middle regions of the elongate body 84.

FIG. 2 shows that the lumen in the body 84 can receive the guidewire 62 in certain embodiments and for certain techniques. The proximal end 80 of the catheter device 58 preferably has a handle 86 that is used to actuate the catheter 58. The handle 86 is configured to transmit a torque. The handle 86 can transmit a torque to the cutting catheter 58. The proximal end 80 can also include a branched access port 88 or other access device. A first branch 90 can be provided to couple with a fluid source F. A second branch 92 can be aligned with the lumen of the cutting catheter 58 to provide in-line access to the lumen in the body 84. One or both of the branches 90, 92 can have a valve structure to limit, minimize or eliminate blood loss. A tuohy-borst attachment can be provided on one or both of the branches 90, 92. In one embodiment, the branched access port 88 can be detached from the handle 86 when access via the branches 90, 92 is not required or for certain phases of procedures where the branches are not needed and might be in the way if not removed from the procedure zone. In one technique, the branched access port 88 is left in place when torquing the catheter 58 because the first branch 90 provides a higher torque than the handle 86 in an optional system and technique.

The distal ends 66, 82 can be configured to be incompressible and/or radiopaque. The distal end 82 can be configured to engage and disrupt the occlusion 20 to enhance access through the stenosis 20. The distal end 82 preferably is stiffer than the elongate body 84 at locations proximal of the distal end 82. The end 82 includes an occlusion clearing implement 94, which can be one or more teeth, a continuous but abrasive surface for removing matter, a concave scooping structure for separating volumes of the matter from the occlusion 17 or other structures discussed herein. As discussed further below, the implement 94 or the system 50 are configured to follow a directed path and not to cause vessel injury in regions not being treated. The implement 94 can be radiopaque to provide visualization of the cutting catheter 58 when disposed in the vasculature.

The sheath 54 is configured to slideably and rotatably receives the catheter device 58. The inner surface of elongate body 65 and/or the outer surface of elongate body 84 can be configured to ease a retracting or extending motion in an axial direction, e.g., along the longitudinal axis of the body 65 or the body 84. Either of these surfaces can have a lubricious coating, for example. In one embodiment, the inner surface of the body 84 includes an expanded polytetrafluoroethylene (ePTFE) or other similar liner. As a result, the end 82 of the cutting catheter 58 can be pulled back into the end 66 of the sheath 54 for delivery or pushed out from the end 66 for engagement with the occlusion 20. The end 66 is configured to minimize out-of-round conditions of the sheath 54. In particular, a support ring 96 of the body 65 can be made more rigid than portions of the elongate body proximal of the distal portion 96 such that the elongate body 84 can freely rotate within the body 65. For example the support ring 96 can include a metal or ceramic cylinder that has hoop strength preventing it from being deformed when urged against an occlusion. The rigidity of the support ring 96 provides the advantage that the distal end 66 will maintain its pre-delivery configuration or will be deformed only by an amount that would not restrict rotation of the body 84 and thereby the end 82. The support ring 96 can be made of a radiopaque material to enhance visibility of the sheath 94 and the system 50.

FIG. 2 also shows that the catheter device 58 can include a second occlusion clearing feature 99 disposed at the proximal end 80. In one embodiment, the second occlusion clearing feature 99 can be disposed within a removeable or moveable housing 99A. The housing 99A can have a clamshell configuration. The housing 99A can be frangible to break along or in specified areas.

In one variation, the clearing implement 94 and the clearing implement 99 can have different cutting characteristics. For example, the clearing implement 94 can be blunt so that the end 82 is configured primarily for supporting advancement of a guidewire or stiff wire through the occlusion. The clearing implement 99 can be active, e.g., including teeth or other sharp features to more rapidly remove an occlusion.

In another variation, the clearing implement 94 and the clearing implement 99 can have the same configuration. This facilitates convenient methods. For example, the first clearing implement 94 can be advanced to an occlusion and used to clear at least a portion thereof. If the lumen in the catheter device 58 near the clearing implement 94 becomes filled, the user can withdraw the catheter device 58. The housing 99A can be removed exposing the clearing implement 99. Thereafter, the clearing implement 99 can be advanced into a blood vessel and up to the occlusion to continue clearing the occlusion. This method can be facilitated by the sheath 54 acting as a guide catheter, as least after the end near the clearing implement 94 is filled.

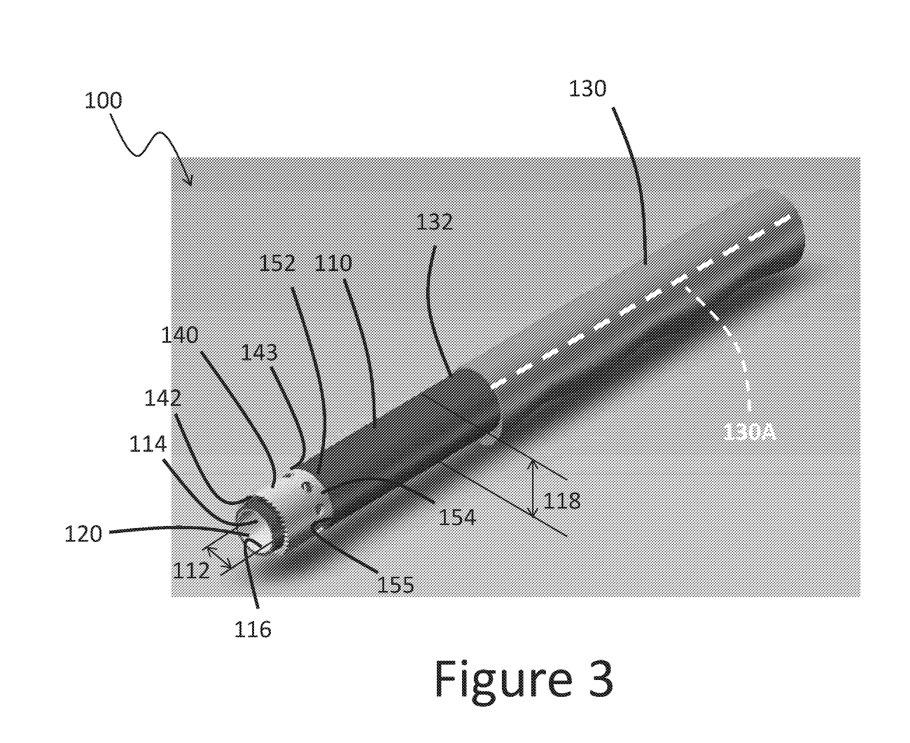

FIG. 3 depicts an embodiment of a clearing device 100. The clearing device 100 as or in combination with the catheter device 58 in the occlusion crossing system 50 discussed above. In the illustrated embodiment, the clearing device 100 has a handle 130 at a proximal end, a tip 140 at a distal end, and a flexible elongate body 110 that is coupled to the handle 130 and the tip 140.

The handle 130 is removable such that the clearing device 100 can be reversed. The clearing device 100 can be reversed such that a tip that is covered by the handle 130 can be exposed and advanced into a blood vessel for an occlusion clearing procedure. The handle 130 can be configured with a seam 130A along which the handle 130 can be opened. The seam 130A can include a hinge on one side of the handle 130 and two opposed edges on a side of the handle 130 oppose the hinge. The opposed edges can include a clasp or hook to hold the edges together. The handle 130 can include a tip space to receive the stowed tip (not shown) or the tip 140 that a can be stowed in the tip space. In certain embodiments, the handle 130 comprises a clam-shell housing. In other embodiments, the handle 130 includes a structure that is configured to preferentially break, e.g., a frangible joint. The frangible joint allows the clinician to quickly break off the handle 130 such that a tip in the tip space can be exposed for advancement into a vessel.

In some embodiments, the elongate body 110 is hollow and cylindrical or substantially cylindrical, having an internal surface 116, a central lumen 114, an inner diameter 112, and an outer diameter 118. In several embodiments, the inner diameter 112 is about 0.94 mm to about 1.07 mm. In several embodiments, the outer diameter 118 is about 1.12 mm to about 1.37 mm. In some embodiments, the central lumen 114 is configured to accommodate a guidewire (not shown). In at least one embodiment, the inner diameter 112 is less than 10% larger than the outer diameter of a guidewire. In other embodiments, a smaller gap on a percentage basis may be provided. For example, some embodiments provide a less than 5% gap between the inner diameter thereof and an outer diameter of a guidewire (e.g., the guidewire 62 of FIG. 2).

In other embodiments and techniques, the guidewire is used to track the clearing device 100 and specifically the tip 140 to the stenosis. Once in position, the guidewire could be withdrawn and the clearing device 100 can be used to enhance access across the occlusion. If the guidewire is in place the clearing device 100 system may rotate about the outer surface of the guidewire independently either exposed in the vessel or in the sheath 54. Thus, in some embodiments, the guidewire is not required to be in place or to rotate with the system for the device to function. In other embodiments and for certain applications, a guidewire may not be used even for delivery of the system. For example, if the vessel segment is straight there may not be a need for a guidewire. In such cases, the clearing device 100 preferably is configured to enhance access across an occlusion without support from a guidewire.

One feature that aids in guidance of the clearing device 100 whether guided by a wire or a guide catheter is the configuration of a rigid distal portion, for example of the tip 140. The tip 140 can be configured to minimize wandering within a blood vessel. In particular, blood is subject to varying pressures and certain peripheral blood vessels have a relatively high mobility. By making the length of the tip 140 greater than inner diameter 112 the distal portion of the clearing device 100 tends to remain generally straight in the vessel. In some embodiments, the length of the tip 140 is more than two times the diameter of the tip. In some embodiments, the length of the tip 140 is more than two and one-half times the diameter of the tip. In some embodiments, the length of the tip 140 is more than three times the diameter of the tip. The tip 140 can be from 1-5 times the diameter of the tip in certain embodiments.

More generally, the clearing device 100 is not limited to natural body lumens or blood vessels. For example, another application for which the clearing device could be used is for salvaging occluded dialysis grafts. Such application may benefit from a lower profile clearing device, e.g., one having an outer diameter of bout 4-8 mm.

In some embodiments, a lining 120 covers at least a portion of the inner surface 116. In some embodiments, the lining 120 is made of a material that enhances the lubricity of the inner surface 116. In at least one embodiment, the lining 120 is made from ePTFE. The lining 120 or other lubricious structure or coating such as silicone or surface modification facilitates sliding of the elongate body 110 over a guidewire in a manner that reduces or minimizes forces that would tend to change the tracking force, the torque force, and the position of a the distal portion, such as a tip of the clearing device 100. As discussed below, in one mode the tip of the clearing device 100 is rotated about the guidewire to provide an abrading or gentle cutting action. Such action could be prevented if the distal portion, e.g., the tip becomes out of round due to such forces.

In several embodiments, the outer surface of the clearing device 100 is coated with a lubricious coating or structure to reduce friction with the vessel wall during tracking, torquing, and crossing of the stenosis. Examples of such structures include a layer of Teflon, silicon, or a hydrophilic coating. A lubricious sleeve could be used, which sleeve can be moveable relative to, e.g., configured to be withdrawn from the clearing device 100.

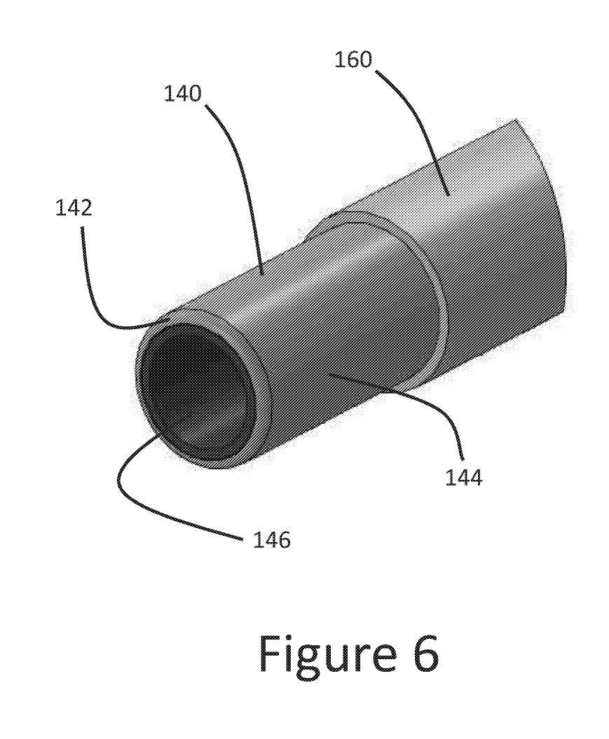

In several embodiments, the clearing device 100 has a tip 140 that is coupled to a distal end 152 of the elongate body 110. In some embodiments, a proximal end 154 of the tip 140 is disposed over the distal end 152 of the elongate body 110. In some embodiments, the distal end 152 of the elongate body 110 is disposed over the proximal end 154 of the tip 140. In at least one embodiment, the proximal end 154 of the tip 140 is coupled face-to-face with the distal end 152 of the elongate body 110 such that the tip 140 and the elongate body 110 share a similar outer diameter, with a proximal face 143 of the tip 140 forming an interface with a distal face 155 of the elongate body 110. In some cases, a transition between the proximal face 143 and a distal end of the elongate provide a joint without a step that could catch on external matter as the device 100 is being delivered. In several embodiments, the cutting tip is attached to the braided skeleton of the catheter body prior to coating the entire structure with an extruded polymer, after which the tip can be subsequently exposed. Other reinforced catheter designs tend to store energy in the reinforcement. The result is something like winding a spring rather than providing one-to-one rotation of the distal end upon rotation of the proximal end. In the catheters herein the braided skeleton is preferably formed to reduce storing energy in the catheter body and to maintain as close as possible one-to-one rotation to enhance the cutting work at the distal end for the rotation at the proximal end. FIG. 6 shows a mesh material 190 to which the tip could be welded or otherwise coupled, for example. In other embodiments, the cutting tip can be attached to the catheter with an adhesive. Variations provide multiple layers of adhesive and layers that can be applied or heat-shrunk over inner layers of the clearing device 10. In some cases, as described herein a recess and/or protrusion provide a strong mechanical interface alone or in combination with other attachment structures.

In several embodiments, the clearing device 100 has a handle 130 coupled to the proximal end 132 of the elongate body 110. In some embodiments, the handle 130 is configured to apply torque to the elongate body 110 as a user rotates the handle 130. In at least one embodiment, the clearing device 100 is configured so that the handle 130 applies an approximately a 1:1 torque ratio to the elongate body 110, causing the tip 140 to rotate substantially in unison with the handle 130. In some embodiments, the handle 130 is made of polymer. In at least one embodiment, the handle 130 is made of polycarbonate.

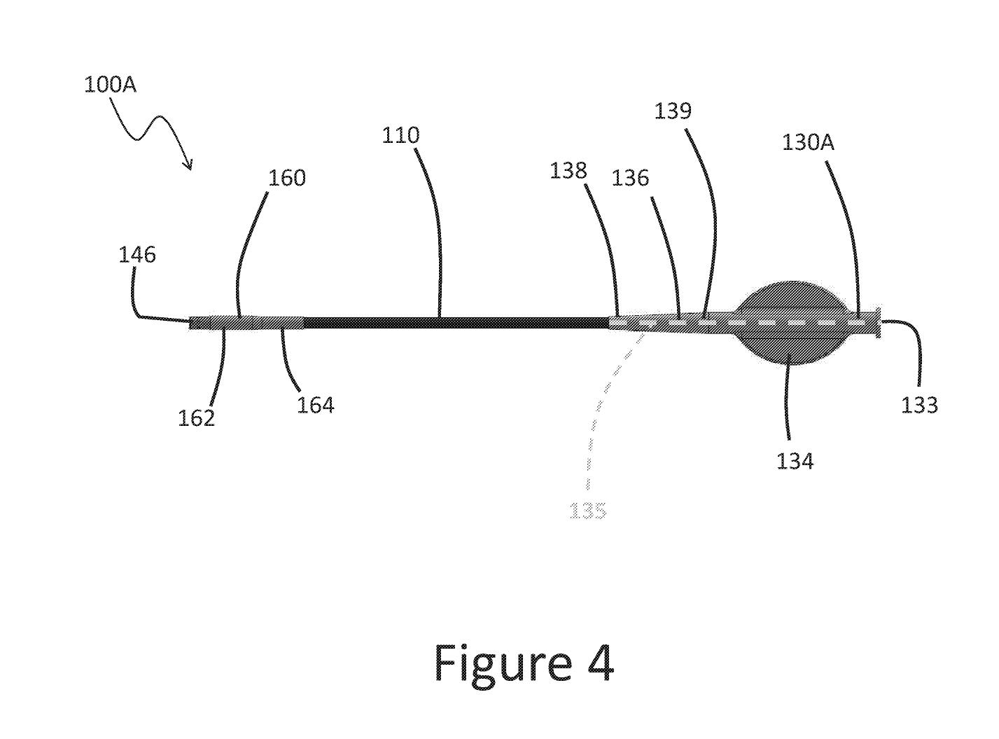

FIG. 4 depicts an embodiment of a clearing device 100A that is similar to the clearing device 100 except as set forth differently below. The clearing device 100A can be used with one or more components of the system 50. In this embodiment, a handle 130A is provided that includes at least one rib 134 that enhances a user's ability to apply torque by finger pressure to the elongate body 110. In the illustrate embodiment, the handle 130A has two ribs that are disposed on opposite sides of the body of the handle 130A. This structure enables a user to apply pressure by a thumb and index finger of a single hand to rotate the clearing device 100A. This provides for easy abrading or cutting action, with the procedure being performed with just one or two hands. For instance, as discussed more below, this approach enables a user to hold a guidewire in one hand and the clearing device 100A in the other hand and to provide rotation of the clearing device over the guidewire when so held.

The handle 130A is configured to be opened or broken to be removed from the device 100A. The handle 130A can have a seam or clam-shell edge 135 that can extend from the proximal end to the distal end of the handle 130A.

In another technique, by altering the tension or compression on the guidewire the user can direct the leading edge of the clearing device 100, 100A. If the wire bows under compression, for example, the trajectory of the clearing device 100 can be altered. A plurality of wires of different bending stiffness could be used to vary the bending stiffness under compression. In one case, two or three wires are provided which will be relatively stiff and could clear cause some enlargement of the occluded lumen. If operation of the clearing device 100 is to commence, one or more of the wires can be removed. For instance a first wire can be removed so that the remaining wires will bow under compression. A tangent to the bent wire(s) that remain will define the trajectory of the clearing device 100, 100A. In a further step, all wires can be removed to permit the clearing device 100, 100A to be advanced without support, and unguided from that point on. For progressive enlargement a series of clearing devices 100 could be used to enlarge the lumen slightly more for each device.

In some embodiments, the handle 130A is joined to the elongate body 110, which is of a different configuration, e.g., a different material or physical structure. In such arrangements, a strain relief structure can be provided between the handle 130A and the elongate body 110 to minimize kinking or other failure modes. One example of a strain relief structure includes a collar 136 that couples handle 130A to the elongate body 110. In at least one embodiment, the collar 136 is bonded to the handle 130A using an adhesive. In some embodiments, the collar 136 is tapered such that a distal end 138 of the collar 136 has an outer diameter that is smaller than the outer diameter of a proximal end 139 of the collar 136. In several embodiments, the collar 136 is made of polymer. In at least one embodiment, the collar 136 is made of nylon. In at least one embodiment, the collar 136 is made of polyether block amide (PEBA). Other functions of the strain relief include one or more of the minimization of kinking during general handling, tracking and torquing of the catheter, facilitating the bonding of a larger diameter handle to the smaller diameter catheter body, providing a surface for the printing of catheter specifications or color to denote the configuration of the catheter. The collar 136 can also have a seam or other structure that enables the collar to be removed to expose a second tip disposed within the handle 130A.

In several embodiments, the clearing device 100 comprises a sleeve 160 that surrounds at least a portion of the elongate body 110. In some embodiments, the sleeve 160 strengthens the junction of the tip 140 to the elongate body 110. In several embodiments, the sleeve 160 minimizes abrupt diameter changes that may result during assembly of the tip 140 to the elongate body 110. In some embodiments, the sleeve 160 surrounds the distal portion of the elongate body 110. In at least one embodiment, the sleeve 160 surrounds the proximal portion of the tip 140 and the distal portion of the elongate body 110. In some embodiments, a distal portion 162 of the sleeve 160 has an outer diameter that is larger than the outer diameter of a proximal portion 164 of the sleeve 160. In at least one embodiment, the sleeve 160 is made of shrink tubing material. Other functions of or modes of operation of the sleeve 160 (e.g., shrink tubing 160) include providing any or all of the following:
lubricity—the outer surface may be made of a material that is more slippery or made to be more slippery than the catheter body thus facilitating tracking and torquing the catheter;
support—the sleeve may be configured to increase the longitudinal stiffness of the distal portion of the catheter, resulting in the cutting tip being guided in a straight; and/or
protection—the sleeve covers the trailing edge of the cutting tip and protects it from being dislodged during tracking and torquing.

Although illustrated as a separate layer that is applied to the elongate body 110, the sleeve 160 could be configured as a coating or could include a coating disposed over it.

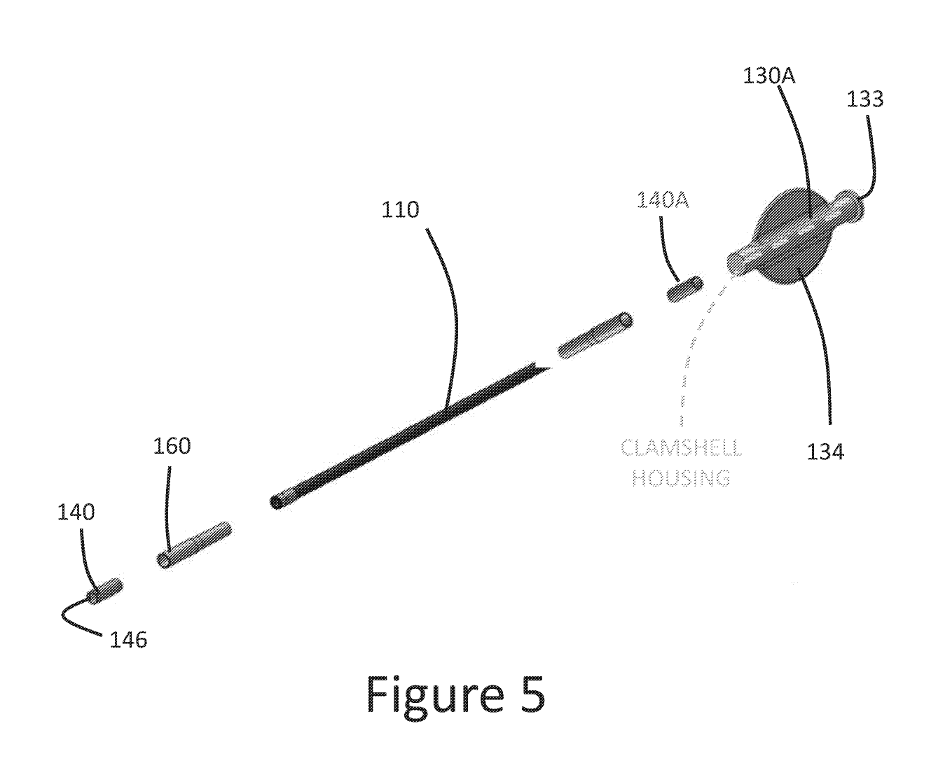

The exploded view of FIG. 5 shows the first and second tips 140, 14A. As discussed more below, the tips 140, 140A are different in their cutting function. The tip 140 can be substantially blunt. In this configuration, the tip 140 can be more focused on supporting a wire while providing gentle abrasion of an occlusion. The tip 140A can be an active tip, e.g., having sharp teeth or cutting features. In one embodiment, the handle 130A initially retains the tip 140A within a tip space while the tip 140 is exposed. In a second mode, the handle 130A can be removed so that the tip 140A can be accessed and used to enhance clearing of an occlusion.

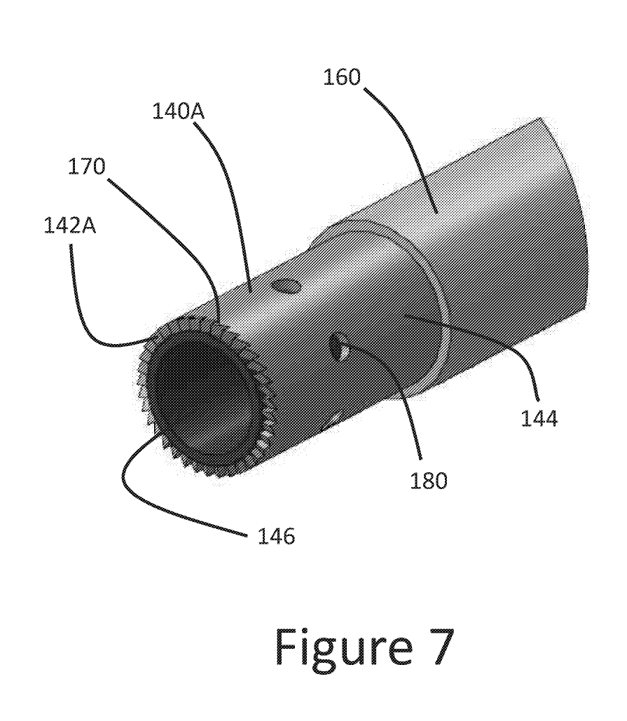

FIGS. 6 and 7 depict illustrative embodiments of the tip 140. As discussed below, the tip 140 interacts with the lesion tissue and is configured to remove or displace lesion tissue. In several embodiments, the tip 140 is configured to remove lesion tissue through different modes of operation including cutting, tearing, shaving, or abrading the lesion tissue. The tip 140 may be configured to use one, or more than one, method of removing lesion tissue. In several embodiments, the tip 140 provides lateral support to the guidewire as the guidewire is advanced through a stenosis. In some embodiments, the tip 140 can prevent the guidewire from buckling as the guidewire is advanced through an occlusion or a near total occlusion. In providing this function, the tip 140 can be configured with a bore having a diameter that is close to that of the guidewire, e.g., within about 10% of the diameter of the guidewire. The gap between the guidewire and the clearing device 100 should be large enough to keep resistance to relative movement (advancement and/or rotation) between these components to an acceptable level for tracking and twisting. In addition, the clearing device 100 may be used as an exchange device for changing guidewires or other interventional devices without losing position or access to the target lesion. The lumen in the clearing device 100 can be used for drug delivery and contrast injection as needed.

The tip 140 has a distal face 142, a side surface 144, and a distal opening 146. In the embodiment of the tip 140 shown in FIG. 6, the distal face 142 of the tip 140 is disposed generally at a plane extending transverse to the longitudinal axis of the tip 140. The face 142 can also be beveled, such that it is rounded in a proximal direction from such a plane, e.g., toward and outer surface of the tip 140. This arrangement is advantageous in that a longitudinal force along the axis of the clearing device 100 will produce a generally straight trajectory of the tip 140 as it advances. The distal face 142 can be disposed on a plane at an acute angle to the longitudinal axis in certain embodiments, but disposing the distal face 142 on a transverse plane results in less deflection of the tip upon advancement or rotation.

In some embodiments the tip 140 can be beveled and serrated. An example of a serrated tip provides a plurality of sharp edges on the surface 140 disposed around the circumference of the tip 140. The sharp edges can be elongated and disposed on the side surface 144. The edges can be axial edges. The edges can be spiral edges. In some embodiments, the sharp edges can be configured for removing material from the clearing zone disposed around the distal face 142. In some embodiments, teeth or other cutting structures can be disposed on the inside of the lumen extending proximally from the distal opening 146. Cutting structures disposed on the side surface 144 can have an arcuate configuration facing the direction of the cut. For example, the cutting surface can have an angle of attack facing the direction of motion of the clearing device. The cutting surface can be position to maximally cut upon rotation of the clearing device 100 in some embodiments. The cutting surface can be positioned to maximally cut upon advancement of the clearing device 100 in some embodiments. In some embodiments, the distal face 142 is blunt (not shown). In at least one embodiment, the distal face 142 is abrasive. In the embodiment of the tip 140A shown in FIG. 7, the distal face 142A has a plurality of cutting teeth 170. The face 142A can also be considered to be disposed on a transverse plane, for example, for example if the distal aspects or proximal aspects of the teeth 170 are disposed at the same plane disposed transverse to the axis of the tip 140A. In some embodiments, the cutting teeth 170 are configured to hold the lesion tissue fixed relative to the tip 140A, allowing the clearing device 100 to tear lesion tissue away from vessel wall. In some embodiments, the cutting teeth 170 are configured to slice through the lesion tissue, allowing the clearing device 100 to remove lesion tissue in a manner that minimizes twisting stress on the vessel wall.

In several embodiments, the side surface 144 of tip 140 includes an element for moving displaced or separated abraded matter from the working zone of the clearing device 100. For example, in one embodiment at least one flute 180 serves to debulk the lesion as the tip 140 rotates within the stenosis 20. In some techniques, aspiration is provided through a main (e.g., central) lumen of the clearing device 100. In some approaches, if a guidewire is present, aspiration through the main lumen can be enhanced by removing the guidewire.

In some embodiments, the flute 180 includes a hole that passes through the tip 140. In at least one embodiment, the flute 180 communicates with a lumen, e.g., a dedicated aspiration lumen (not shown) or the central lumen 114 of the elongate body 110. If the abraded matter is to be aspirated out of the clearing device 100, 100A or otherwise segregated therein, a dedicated lumen may be preferred in that the sliding contact between the inner surface 116 of the elongate body 110 and the outer surface of the guidewire should remain as debris-free as possible to reduce the chance of these surfaces becoming seized. In other embodiments, a greater gap is provided between the inside surface of the elongate body 110 and a guidewire positioned therein and abraded or separated matter from the occlusion can be aspirated or sequestered in the main lumen. In at least one embodiment, the flute 180 is a circular hole having a diameter of 6.6 mm.

Other uses for the flutes 180 are to confirm the status of the clearing device 100. For example, an imaging agent can be delivered through a lumen in fluid communication with the flutes. The pattern of the images indicates the status of the clearing device. In one instance, the imaging agent may not emerge from the clearing device 100. The clinician can then know that the clearing device 100 is occluded and could be removed and either cleared or replaced with a second clearing device. In another instance, the imaging agent can indicate whether the occlusion of the vessel is sufficiently enlarged for other treatment. In another instance, the imaging agent may indicate that a different mode of use of the clearing device 100 should be used. For example, if one side of the clearing device 100 is occluded a second side of the device could be rotated into position to further clear the lumen.

In some embodiments, apertures similar to the flutes 180 can be provided through the tip 140A to provide for securement to other parts of the clearing device. For example, the tip 140A can be configured as a metal cylinder to be joined to an elongate polymeric catheter body. To secure the cylinder, holes in the cylinder can be configured and positioned to have portions of the catheter flow or extend into the holes. In one technique a polymeric body of the clearing device 100 disposed on the inside of the cylinder is formed such that a portion thereof protrudes radially outwardly into the holes. In one technique a polymeric body of the clearing device 100 disposed on the outside of the cylinder is formed such that a portion thereof protrudes radially inwardly into the holes. FIG. 5A shows an example where holes are disposed beneath the sleeve 160. The sleeve is applied to a portion of the side surface 144 of the tip 140A such that the sleeve extends into the holes. This provides for securement of the cylinder to prevent it from slipping off the elongate body 110 or from being displaced proximally which would interfere with the clearing function.

The elongate body 110 proximal of the tip 140 must be flexible to enable the clearing device 100 to travel through a tortuous vasculature for certain applications, e.g., for coronary or neurovascular procedures. At the same time, the elongate body 110 must be stiff to transmit compressive and torsional forces to the tip 140. In several embodiments, the elongate body 110 is made of polymer. In some embodiments, the elongate body 110 is made of a polymeric material selected from the group consisting of polyimide, and PEBA. In some embodiments, the elongate body 110 is made of one material embedded in another material. The elongate body 110 can be made of a mesh material embedded in a coat material. In at least one embodiment, the mesh material includes 304-stainless steel flat wire braid. In at least one embodiment, the coat material is made of a polymer, such as polyimide and/or PEBA. In some embodiments, multiple layer and multiple polymers may be employed. In addition, in several embodiments the elongate body can be fashioned from a material or composite structure at the proximal end to provide more push such as a hypotube and be attached to a material of softer stiffness to facilitate tracking and delivery of the cutting tip.

In several embodiments, the clearing device 100 is used in conjunction with a guidewire (not shown). In many embodiments, a guidewire is advanced endovascularly until the distal end of the guidewire reaches a stenosis targeted for angioplasty. In some embodiments, the clearing device 100 is mounted onto a guidewire by feeding the proximal end of the guidewire into the distal opening 146 of the tip 140. The guidewire is then passed through the central lumen 114 of the capture device 110, and drawn out of the proximal opening 133 of the handle 130. The tip 140 is advanced along the guidewire until the distal face 142 of the tip 140 encounters the lesion 17. As discussed elsewhere herein, close clearance between the lumen 114 and a guidewire help to reduce the crossing profile. In some embodiments and in some techniques, having close clearing also helps in bracing the guidewire. Bracing is not required for various embodiments and techniques. In some cases, a guidewire is not needed in any aspect of the method.

In some embodiments, the tip 140 is used to gently abrade the lesion 17. Once the distal face 142 makes contact with the lesion 17, a user applies torque to the handle 130, causing the handle 130 to rotate about the guidewire. The elongate body 110 transmits the torque to the tip 140, causing the distal face 142 of the tip 140 to slide over the surface of the lesion 17. In many embodiments, a user rotates the handle 130 in alternating clockwise and counterclockwise directions. In some embodiments, the handle 130 is rotated in only one direction. In some embodiments, a user applies compressive forces by pushing the handle 130 in the distal direction. In some embodiments, a user applies simultaneously compressive and torsional forces by pushing the handle 130 in the distal direction while rotating handle 130 about the guidewire.

In several embodiments, the tip 140 is configured to resist deformation. In some embodiments, the tip 140 is made of an alloy possessing high strength properties. In at least one embodiment, the tip 140 is made of seamless drawn tubing of L-605 composition. In several embodiments, the tip 140 defines a circular lumen. In at least one embodiment, the tip 140 has an inner diameter of 1.25 mm and a circularity of less than 0.0050 mm. In at least one embodiment, the tip 140 is a hollow cylinder with an outer diameter of 1.45 mm, a wall thickness of 0.2 mm, and a length of 4.5 mm. As noted above, configuring the tip to avoid being deformed out of round provides assurance that the clearing device 100, 100A will not seize up upon the guidewire, preventing relative rotation. In at least some embodiments, a close fit between the guidewire and the clearing device 100, 100A is provided so that the clearing device 100 can provide a bracing effect to the guidewire. This bracing effect enables the guidewire to be advanced distally out of the abrading device in a mode of operation where the guidewire is urged forward across the lesion. In order to provide this bracing effect while still maintaining the clearing device 100 rotatable over the guidewire, out of round of the inner diameter should be reduced, minimize or eliminated.

Further methods of using the occlusion crossing system 50 or devices 100, 100A or similar systems with any of the alternative components described herein are discussed below and in PCT Application No. PCT/US2014/056162, filed Sep. 17, 2014, which is incorporated by reference herein in its entirety.

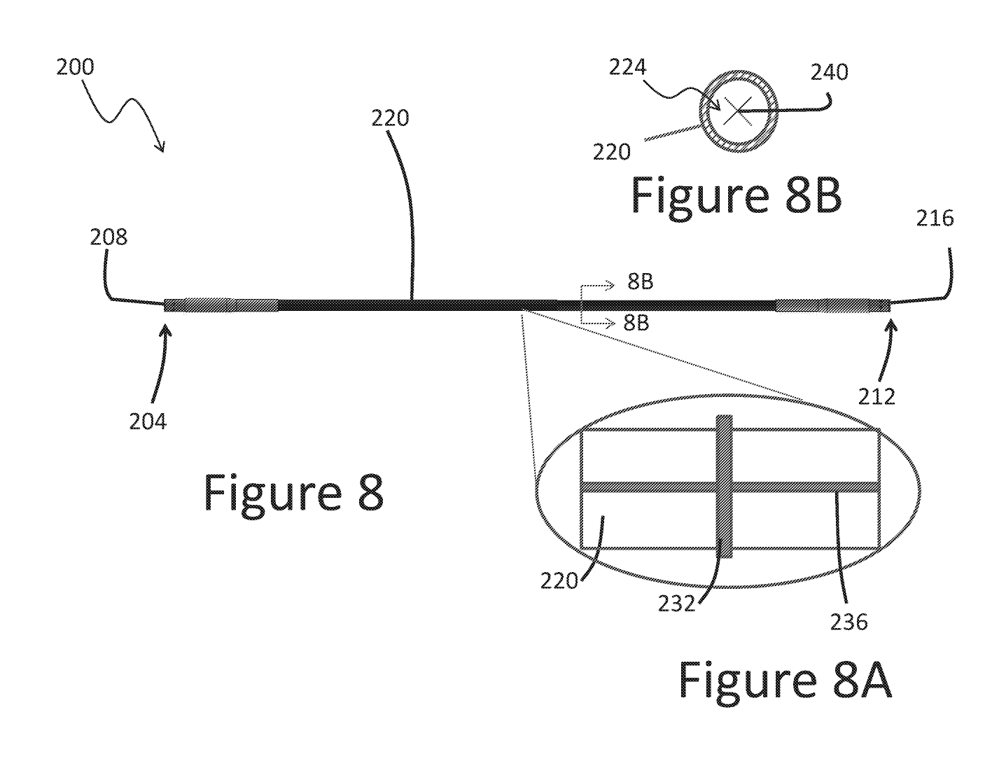

FIGS. 8-8B illustrate a dual end occlusion crossing device 200. The device includes a first end 204 that has a first occlusion engaging feature 208 disposed at a free end. A second end 212 has a second occlusion engaging feature 216 disposed at a free end thereof. An elongate body 220 extends between the first end 204 and the second end 208. The elongate body 220 has a lumen 224 that extends therethrough. The device 200 is dual ended, in that both the first and second ends 204, 212 can be advanced into a patient to act on an occlusion as discussed above.

In various methods, the first end 204 can be advanced first to the occlusion and act thereon. Thereafter the first end 204 can be removed from the patient and the second end 212 can be advanced into the patient to act on the occlusion.

In the illustrated embodiment, the device 200 includes a stop member 232 slideably mounted to a side portion of the elongate body 220. The stop member 232 is slideable in a track 236 that extends between a first end disposed toward the first end 204 of the device 200 and a second end disposed toward the second end 216 of the device 200. The track allows the stop member 232 to be manually shifted from a first location where the majority of the length of the elongate body 220 is between the stop member 232 and the first end 204 to a second location where the majority of the length of the elongate body 220 is between the stop member 232 and the second end 212. When the stop member 232 is in the first location, the first end 204 can be advanced in the vasculature to a location adjacent to the occlusion. When the stop member 232 is in the second location, the second end 216 can be advanced in the vasculature to a location adjacent to the occlusion. The stop member 232 is configured to abut an introducer sheath when the device 200 is fully inserted so that the device 200 cannot be inadvertently advanced fully into the patient. For example, the stop member 232 can have a diameter that is greater than the inner diameter of the introducer sheath.

FIG. 8B shows that in some embodiments a valve 240 is provided in the lumen 224 of the elongate body 220. The valve 240 can take any suitable form, but preferably is configured to reduce or minimize or eliminate blood loss when the device 200 is placed in the vasculature. The valve 240 can be configured to fit snugly around a guidewire while impeding blood loss between the guide wire and the lumen. The valve 240 can be placed anywhere along the length of the elongate body 220. In one embodiment the valve is placed midway between the first end 204 and the second end 212 so that the valve characteristic are similar whether the first end or the second end is inserted. In another embodiment, the valve 240 is coupled with the stop member 232 so that the valve 240 is near the end disposed outside the patent. The valve 240 can even be located outside the patient. In one embodiment, the valve allows some blood to flow backwards to allow the distal end to accommodate particles generated at the end in contact with the occlusion.

Other features described herein can be combined with the device 200. For example, the sharp and abrasive tips of FIGS. 6 and 7 can be incorporated at one or both of the first and second ends 204, 212. Torque enhancing features such as the ribs 134 could be included on the slideable ring 232. These are examples of the many features that can be included form the embodiments discussed above in the dual end occlusion crossing device 200.

In another embodiment, a method is provided for enhancing access across an occlusion. A first end of a catheter is advanced into a blood vessel and up to a proximal face of an occlusion. The size of a lumen across at least a portion of an occlusion is expanded by engaging the first end of the catheter with the occlusion. The catheter is removed from the patient. A second end of the catheter opposite the first end is advanced into the blood vessel and up to the proximal face of the occlusion. The size of a lumen across at least a portion of the occlusion is further expanded by engaging the second end of the catheter with the occlusion.

The insertion of the catheter can involve positioning a guidewire through a valve in the catheter.

Between inserting the first end and the second end, a stop member can be moved along, e.g. slid along, an outside surface of the catheter.

The above presents a description of modes contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiments disclosed. On the contrary, the intention is to cover modifications and alternate constructions coming within the spirit and scope of the invention as generally expressed by the following claims, which particularly point out and distinctly claim the subject matter of the invention.

What is claimed is:

1. A catheter device, comprising:
a first end having a first rigid ring disposed thereon, the first rigid ring having an occlusion engaging feature disposed at a free end thereof;
a second end having a second rigid ring disposed thereon, the second rigid ring having an occlusion engaging feature disposed at a free end thereof;
an elongate body extending between the first end and the second end, the elongate body having a lumen extending therethrough,
a handle being configured such that a body thereof can be coupled with the elongate body at a first position adjacent to the first end and such that the second end can be advanced into a patient, the inner diameter of the second rigid ring configured for sliding and supporting interaction with a guidewire;
the handle being configured such that the body thereof can be coupled with the elongate body at a second position adjacent to the second end and such that the first end can be advanced into a patient, the inner diameter of the first rigid ring configured for sliding and supporting interaction with a guidewire.

2. The catheter of claim 1, wherein the handle comprises a clamshell configuration such that body can be opened to provide access to a space configured to retain a portion of the elongate body.

3. The catheter of claim 1, wherein the handle comprises a compression fitting that can be opened to permit the handle to slide off one end and that can be closed to retain a portion of the elongate body in a space within the that body of the handle.

4. A dual end occlusion crossing device, comprising:
a first end having a first occlusion engaging feature disposed at a free end thereof;
a second end having a second occlusion engaging feature disposed at a free end thereof;
an elongate body extending between the first end and the second end, the elongate body having a lumen extending therethrough.

5. The dual end occlusion crossing device of claim 4, further comprising a stop member slideably mounted to a side portion of the elongate body.

6. The dual end occlusion crossing device of claim 4, further comprising a valve disposed in the lumen of the elongate body.

7. The dual end occlusion crossing device of claim 4, further comprising a rigid ring disposed at the first end, the rigid ring comprising the first occlusion engaging feature.

8. The dual end occlusion crossing device of claim 5, further comprising a second rigid ring disposed at the second end, the second rigid ring comprising the second occlusion engaging feature.

9. The dual end occlusion crossing device of claim 4, further comprising a handle configured such that a body thereof can be coupled with the elongate body at a first position adjacent to the first end and such that the second end can be advanced into a patient, the inner diameter of the second end configured for sliding and supporting interaction with a guidewire.

10. The dual end occlusion crossing device of claim 7, wherein the handle is configured such that the body thereof can be coupled with the elongate body at a second position adjacent to the second end and such that the first end can be advanced into a patient, the inner diameter of the first end configured for sliding and supporting interaction with a guidewire.

11. A method, comprising:
advancing a first end of a catheter into a blood vessel and up to a proximal face of an occlusion;
expanding the size of a lumen across at least a portion of an occlusion by engaging the first end of the catheter with the occlusion;
removing the catheter from the patient;
advancing a second end of the catheter opposite the first end into the blood vessel and up to the proximal face of the occlusion;
further expanding the size of a lumen across at least a portion of the occlusion by engaging the second end of the catheter with the occlusion.

12. The method of claim 11, further comprising moving a handle of the catheter from a location adjacent to the second end to a location adjacent to the first end.
